(19) United States
(12) Patent Application Publication
(10) Pub. No.: US 2018/0071248 A1
(43) Pub. Date: Mar. 15, 2018

(54) COMPOSITION FOR IMPROVING CIRCADIAN RHYTHM

(71) Applicant: SUNTORY HOLDINGS LIMITED, Osaka (JP)

(72) Inventors: Norifumi TATEISHI, Kyoto (JP); Shigenobu SHIBATA, Tokyo (JP)

(73) Assignee: SUNTORY HOLDINGS LIMITED, Osaka (JP)

(21) Appl. No.: 15/560,277

(22) PCT Filed: Mar. 22, 2016
(86) PCT No.: PCT/JP2016/058963

§ 371 Date: Sep. 21, 2017

(30) Foreign Application Priority Data
Mar. 23, 2015 (JP) 2015-059753

(51) Int. Cl. A61K 31/36

(52) U.S. Cl. CPC A61K 31/36

(57) ABSTRACT

The purporse of the present invention is to provide a circadian rhythm improving composition which can be safely taken over a long time, in particular, a circadian rhythm controlling composition capable of increasing circadian rhythm amplitude. Provided is a circadian rhythm improving composition comprising one or more kindes of lignan compounds.

### TECHNICAL FIELD

The present invention relates to a composition for improving circadian rhythm. More particularly, this invention relates to a composition for improving circadian rhythm based on amplification of the amplitude of periodic expression of clock genes, the composition comprising a lignan compound.

### BACKGROUND ART

Many biological activities and physiological phenomena exhibit autonomous, periodic changes called biorhythms. Among them, those rhythms which show a periodicity of about 24 hours are called “circadian rhythms.” Examples of biological activities and physiological phenomena with circadian rhythms include sleep and awakening, variation in blood pressure, variation in body temperature, and eating behavior.

All cells constituting the body each have the mechanism of exhibiting circadian rhythm. This circadian rhythm exhibiting mechanism is based on variation of expression of a group of genes called clock genes at a 24-hour cycle. Each of cells works in sync with each other to exhibit circadian rhythm, and thereby circadian rhythms in biological activities and physiological phenomena take place at the tissue, organ and organism levels. There are three indicators for defining the rhythmicity of clock gene expression: period length (length of one period, which is about 24 hours), phase (peak time of expression), and amplitude (range of variation in expression level). Clock genes exhibit a large variation in gene expression level, i.e., a large amplitude of expression, between day and night. An organism's behavioral patterns, which vary between day and night, are achieved through repetition of such a variation in clock gene expression at a 24-hour cycle. In contrast, it is known that abnormal conditions of at least one of period length, phase and amplitude disturb circadian rhythms in biological activities. Disturbance of circadian rhythms may cause bad health conditions or their associated diseases, such as sleep disorders, autonomic imbalance, endocrine disorders, or lifestyle diseases.

In the super-aging society in recent years, there are problems of flattening of behavioral patterns between day and night, including fragmented sleep or very early morning awakening in elderly people. Such problems are believed to be associated with circadian rhythms. It has been observed in aged animal studies that aged animals experience changes in circadian rhythms at the behavioral level, such as decline in sleep quality or reduction in activity amount at the active period. Also, it has recently been suggested that there is a relationship between aging and abnormality in clock genes (NPLs 1 to 3). For example, animals deficient in clock genes like Bmal and CLOCK were found to be prematurely aged. Further, it has been reported that control of the clock oscillation mechanism is disrupted with aging at the cellular level, and that flattening of the amplitude of circadian rhythm is observed particularly in senescent cells and thus may be involved in the aging of organisms.

There is a need for a circadian rhythm controlling composition capable of improving such abnormability of circadian rhythms, in particular, a circadian rhythm controlling composition capable of amplifying the amplitude of circadian rhythm. Such a circadian rhythm controlling composition is useful for improving sleep disorders such as fragmented sleep or very early awakening, and ameliorating bad health conditions of people experiencing fatigue on a daily basis, such as difficulty falling asleep, decreased depth of sleep, and difficulty awakening.

As for a circadian rhythm controlling agent which is focused on the secretion rhythm of melatonin, a hormone related to circadian rhythm, there is a report of a circadian rhythm controlling agent comprising whey as an active component (PTL 1). As for circadian rhythm controlling agents which are focused on circadian rhythms in clock gene expression, there were reports relating to the following: a circadian rhythm controlling agent comprising a plant alkanoid as an active component, wherein the circadian rhythm controlling agent is capable of controlling the period length of the circadian rhythm in expression of the clock gene Bmal 1 (PTL 2); a circadian rhythm controlling agent comprising a Lactobacillus fermentation product as an active component, wherein the circadian rhythm controlling agent is capable of increasing the expression levels of the clock genes Rev-erbα and PPARα (PTL 3); a circadian rhythm controlling agent comprising a Lactobacillus bacterium as an active component, wherein the circadian rhythm controlling agent is capable of controlling the phase of expression of the clock genes Per 1, Per 2, and Bmal 1 (PTL 4); and a circadian rhythm controlling agent comprising a flavonoid and the like as active components, wherein the circadian rhythm controlling agent is capable of controlling the phase of circadian rhythm by suppressing expression of the Bmal 1 gene (PTL 5). However, none of the foregoing agents is focused on the amplitude of clock gene expression rhythm.

NPLs 4 and 5 disclose that caffeine has activities to amplify circadian rhythm amplitude and to lengthen circadian rhythm period. However, caffeine is problematic in terms of the safety of excessive intake.

### CITATION LIST

### Patent Literatures

PTL 1: International Patent Publication No. WO 2005/094849

PTL 2: Japanese Patent Application Publication No. JP 2011-37755

PTL 3: Japanese Patent Application Publication No. JP 2008-179573

PTL 4: Japanese Patent Application Publication No. JP 2013-181005

PTL 5: Japanese Patent Application Publication No. JP 2008-266319

### Non Patent Literatures

NPL 1: Cell, 2013, 153, 1421-1422

NPL 2: Frontiers in Genetics, 2015, 5(455), 1-7

NPL 3: Aging and Disease, 2014, 5(6), 406-418

NPL 4: Biochem Biophys Res Commun. 2011, 410(3), 654-8

NPL 5: Br J Pharmacol. 2014, 171(24), 5858-69

### SUMMARY OF INVENTION

### Technical Problem

In order to solve the problem of flattening of behavioral patterns between day and night, which is considered problematic particularly in the life of elderly people, it is necessary to bring the amplitude of variation in clock gene expression levels into its proper, highly variable state. By bringing the amplitude of clock gene expression levels into a highly variable state, bad health conditions such as difficulty falling asleep, decreased depth of sleep, and difficulty awakening can be ameliorated. An object of the present invention is to provide a circadian rhythm improving composition which can be safely consumed for a long time, in particular, a circadian rhythm controlling composition capable of amplifying circadian rhythm amplitude.

### Solution to Problem

The present inventors have made intensive studies to solve the aforementioned problems, and as a result, found that lignan compounds present in sesame, a food with a long history of being eaten, have an activity to amplify the amplitude of clock gene expression rhythm.

More specifically, the present invention preferably includes, but is not limited to, the following embodiments.
[1] A circadian rhythm improving composition comprising at least one lignan compound.
[2] The circadian rhythm improving composition as set forth in [1], wherein the lignan compound comprises sesamin, episesamin, sesamolin, sesamol, or sesaminol, or a glycoside or metabolite thereof, or a mixture of two or more thereof.
[3] The circadian rhythm improving composition as set forth in [1] or [2], wherein the circadian rhythm improving composition is capable of amplifying the amplitude of the expression rhythm of at least one clock gene.
[4] The circadian rhythm improving composition as set forth in [3], wherein the clock gene comprises Per 2 or Cry 1 gene.
[5] The circadian rhythm improving composition as set forth in any one of [1] to [4], for use in the prevention, alleviation or treatment of a circadian rhythm disorder associated with aging.
[6] The circadian rhythm improving composition as set forth in [5], wherein the circadian rhythm disorder associated with aging is selected from the group consisting of sleep disorders, day-night reversal of behaviors, and flattening of body temperature rhythm amplitude, which are associated with aging.
[7] The circadian rhythm improving composition as set forth in any one of [1] to [4], for use in the amelioration of difficulty falling asleep, decreased depth of sleep, and difficulty awakening.
[8] The circadian rhythm improving composition as set forth in any one of [1] to [7], wherein the circadian rhythm improving composition is provided as an agent.
[9] A process for producing a composition having circadian rhythm improving activity, the process comprising the step of formulating at least one lignan compound.
[10] The process as set forth in [9], wherein the lignan compound comprises sesamin, episesamin, sesamolin, sesamol, or sesaminol, or a glycoside or metabolite thereof, or a mixture of two or more thereof.
[11] Use of at least one lignan compound in the production of a composition having circadian rhythm improving activity.
[12] The use as set forth in [11], wherein the lignan compound comprises sesamin, episesamin, sesamolin, sesamol, or sesaminol, or a glycoside or metabolite thereof, or a mixture of two or more thereof.

### Advantageous Effects of Invention

The circadian rhythm improving composition of the present invention comprises at least one lignan compound, and thereby is capable of amplifying the amplitude of clock gene expression rhythm and improving circadian rhythm. The circadian rhythm improving composition of this invention makes it possible to effectively prevent, alleviate, or treat circadian rhythm disorders associated with flattening of the amplitude of clock gene expression rhythm, in particular, circadian rhythm disorders associated with aging, and to ameliorate difficulty falling asleep, decreased depth of sleep, and difficulty awakening.

### BRIEF DESCRIPTION OF DRAWINGS

FIG. 1 shows a model diagram of the period of clock gene expression.

FIG. 2 shows the effect of administration of a 1:1 mixture of sesamin/episesamin to a Per 2::LUC knock-in mouse-derived embryonic fibroblast line on the amplitude of the Per 2 gene expression rhythm.

FIG. 3 shows the effect of oral administration of a 1:1 mixture of sesamin/episesamin to Per 2::LUC knock-in mice on the amplitude of the Per 2 gene expression rhythm in different organs of the mice.

FIG. 4 shows the effect of administration of each of sesamin, episesamin and sesaminol to a Per 2::LUC knock-in mouse-derived embryonic fibroblast line on the amplitude of the Per 2 gene expression rhythm.

### DESCRIPTION OF EMBODIMENTS

<Lignan Compound>

The circadian rhythm improving composition of the present invention comprises at least one lignan compound. Examples of the lignan compound used in this invention include sesamin, sesaminol, episesamin, episesaminol, sesamolin, 2-(3,4-methylenedioxyphenyl)-6-(3-methoxy-4-hydroxyphenyl)-3,7-dioxabicyclo[3,3,0]octane, 2,6-bis-(3-methoxy-4-hydroxyphenyl)-3,7-dioxabicyclo[3,3,0]octane, 2-(3,4-methylenedioxyphenyl)-6-(3-methoxy-4-hydroxyphenoxy)-3,7-dioxabicyclo[3,3,0]octane, and the like, and metabolites or glycosides thereof. Such compounds can be used alone or in combination. For the purpose of this invention, sesamin, episesamin, sesamolin, sesamol, or sesaminol, or a glycoside or metabolite thereof, or a mixture of two or more thereof can be advantageously used.

The aforementioned lignan compounds are by no means limited in terms of their form, production process, or the like. For example, an extract (lignan compound-rich extract or purified product) obtained from sesame oil by a known method (e.g., a method in which sesame oil is subjected to extraction by addition of hot methanol, and after removal of methanol, the extraction residue is subjected to further extraction by addition of acetone (as described in Japanese Patent Application Publication No. JP H04-9331)) may be used. Also, a lignan compound can be obtained synthetically. To cite some examples of the synthetic methods, sesamin and episesamin can be synthesized by the method of Beroza, et al. (J. Am. Chem. Soc., 78, 1242 (1956)), pinoresinol can be synthesized by the method of Freundenberg, et al. (Chem. Ber., 86, 1157 (1953)), and syringaresinol can be synthesized by the method of Freundenberg, et al. (Chem. Ber., 88, 16 (1955)).

When a lignan compound concentrate extracted and/or purified from a food-derived ingredient such as sesame oil is used as the lignan compound used in the present invention, the degree of concentration should be selected as appropriate depending on the type of the lignan compound and the form of the circadian rhythm improving composition to be formulated. For example, it is generally preferred to use a lignan compound concentrate which is concentrated to give a total lignan compound content of not less than 1 wt. %. The total lignan compound content in the lignan compound concentrate is preferably not less than 20 wt. %, more preferably not less than 50 wt. %, still more preferably not less than 70 wt. %, most preferably not less than 90 wt. %.

<Circadian Rhythm Improving Composition>

The circadian rhythm improving composition of the present invention comprises at least one lignan compound, and thereby is capable of amplifying the amplitude of expression rhythm of at least one clock gene and improving circadian rhythm.

Clock genes are a group of genes that function to control circadian rhythms, and are defined as genes that affect behavioral rhythms when they are mutated (Ishida, 2009, Seikagaku (Biochemistry), vol. 81, no. 2, p. 75-83). Examples of clock genes include, but are not limited to, Period genes, Bmal gene, Clock gene, and Cryptochrome genes. The circadian rhythm improving agent of the present invention is capable of amplifying the amplitude of expression rhythm of preferably at least one of the genes belonging to the Period gene family and the genes belonging to the Cryptochrome gene family, and more preferably at least one of Period 1 (Per 1) gene, Period 2 (Per 2) gene, Period 3 (Per 3) gene, Cryptochrome 1 (Cry 1) gene, and Cryptochrome 2 (Cry 2) gene.

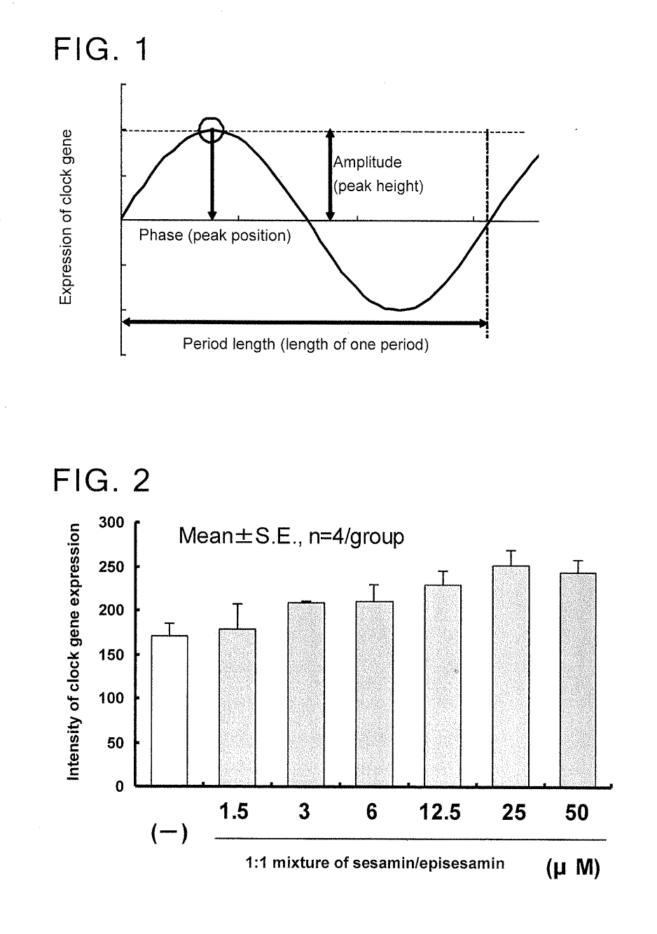

The expression rhythm of clock genes refers to a periodic variation in expression levels as observed by monitoring over time changes in clock gene expression level. FIG. 1 shows a model diagram of the period of clock gene expression. As shown in this figure, there are three indicators for defining the rhythmicity of clock gene expression rhythm: period length (length of one period, which is about 24 hours), phase (peak position of expression, which is represented by peak time of expression), and amplitude (range of variation in expression level). By stating that the amplitude of clock gene expression rhythm is amplified by the circadian rhythm improving composition of the present invention, it is meant that the difference between the highest and lowest clock gene expression levels is made larger than that observed before using said inventive composition or when not using said inventive composition—in other words, the range of variation in clock gene expression level is widen. Amplifying the amplitude of clock gene expression rhythm results in improvement in circadian rhythms.

Examples of the improvement in circadian rhythms include, but are not limited to: restoration of the circadian rhythm in metabolism or secretion in cells to or close to normal; restoration of the circadian rhythm in metabolism or secretion in tissues to or close to normal; and restoration of the circadian rhythms in behavioral or physiological responses in an organism to or close to normal. Since the circadian rhythm improving composition of the present invention is capable of amplifying the amplitude of clock gene expression rhythm, said inventive composition can be advantageously used particularly for the prevention, alleviation or treatment of circadian rhythm disorders associated with flattening of clock gene expression amplitude. Examples of circadian rhythm disorders associated with flattening of clock gene expression amplitude include sleep disorders, day-night reversal of behaviors, and flattening of body temperature rhythm amplitude. Other examples of circadian rhythm disorders associated with flattening of clock gene expression amplitude include, but are not limited to, circadian rhythm disorders associated with aging, and difficulty falling asleep, decreased depth of sleep, and difficulty awakening.

The composition of the present invention can be provided in the form of, for example, an agent, but this form is not the sole example. Such an agent can be provided directly as a composition or can be provided as a composition comprising said agent. Examples of such a composition include, but are not limited to, pharmaceuticals (pharmaceutical compositions), food and beverage compositions (e.g., beverage products, food products), and cosmetics (cosmetic compositions). Non-limiting examples of food compositions include functional foods, health supplementary foods, foods with nutrient function claims, special use foods, foods for specified health use, nutritional supplements, foods for dietary cure, health foods, and dietary supplements.

The circadian rhythm improving composition of the present invention can be applied to both therapeutic use (medical use) and non-therapeutic use (non-medical use). To be specific, said inventive composition can be used as a pharmaceutical, a quasi-drug, a cosmetic, or the like, or as a composition that does not belong to any of the foregoing under the phaiinaceutical affairs law but which explicitly or implicitly targets for prevention, alleviation, treatment or the like of circadian rhythm disorders associated with flattening of clock gene expression amplitude. Said inventive composition can also be provided as a composition with an explicit indication of its efficacy, and can for example bear a label containing information related to “ameliorating sleep disorders”, “improving sleep quality”, “ameliorating difficulty falling asleep”, “ameliorating day-night reversal”, “ameliorating flattening of body temperature rhythm amplitude”, “ameliorating difficulty awakening”, “recovery from fatigue”, and the like. To cite a specific example, said inventive composition can bear a label based on its circadian rhythm improving effect, which states, for example, “Helps to ameliorate bad health conditions of people experiencing fatigue on a daily basis, such as difficulty falling asleep, decreased depth of sleep, and difficulty awakening.”

The expression level of clock genes upon using the circadian rhythm improving composition of the present invention can be analyzed using a given method known to those skilled in the art. Examples of such a known method that can be advantageously used include expression level analyses using Northern blotting analysis, realtime RT-PCR analysis, and DNA microarray, which use a certain sequence region in a clock gene sequence of interest as a probe. A sample that can be used for gene expression analysis can be of any type as long as the sample contains cells, and examples of the sample that can be used include hair containing hair follicle cells, biopsies of skin and like, blood, and saliva.

The circadian rhythm improving composition of the present invention can, if necessary, have mixed therewith any other additives such as mineral; vitamins like vitamin E, C and A; nutritional ingredients; flavorants; and colorants, as long as such additives do not impair the effects of a lignan compound, namely, mixing a lignan compound with such additives does not give rise to an unfavorable interaction.

The form of the circadian rhythm improving composition of the present invention is not particularly limited, and said inventive composition can be formulated into various pharmaceutical dosage forms, including liquid preparations such as solution, suspension and emulsion; semi-solid preparations such as paste; granules; powdered medicines; powders; tablets; and capsules (including soft and hard capsules). The circadian rhythm improving composition of this invention can also be used as an internal composition for use in capsules.

The amount of a lignan compound to be included as an active component in the circadian rhythm improving composition of the present invention can be determined as appropriate in consideration of its effects. For example, when sesamin is selected as a lignan compound, the circadian rhythm improving composition of this invention can comprise the lignan compound in a total amount of 0.001 to 90 wt. %, 0.01 to 50 wt. %, or 0.1 to 10 wt. %.

The dose and mode of administration of the circadian rhythm improving composition of the present invention can be selected as appropriate in consideration of the age, body weight, conditions, and the like of a subject. For example, when sesamin selected as a lignan compound is to be orally administered to a human (adult), it is generally advisable to sequentially administer said inventive composition about once or twice daily, at a rate of 5 or more times per week, so as to give a daily dose of sesamin of about 1 to 200 mg, preferably about 3 to 100 mg, more preferably about 5 to 50 mg.

<Process for Producing a Composition Having Circadian Rhythm Improving Activity>

The present invention also relates to a process for producing a composition having circadian rhythm improving activity, the process comprising the step of formulating at least one lignan compound.

The form of the composition of the present invention is not particularly limited, and examples of the form include pharmaceutical compositions such as pharmaceuticals and quasi-drugs, as well as compositions that do not belong to any of the foregoing under the pharmaceutical affairs law but which, like pharmaceuticals and quasi-drugs, explicitly or implicitly target for prevention, alleviation or treatment of circadian rhythm disorders or amelioration of difficulty falling asleep, decreased depth of sleep, or difficulty awakening with the aim of bringing life and behavioral patterns into a highly variable state between day and night. Such compositions include food compositions, and non-limiting examples of the food compositions include functional foods, health supplementary foods, foods with nutrient function claims, special use foods, foods for specified health use, nutritional supplements, foods for dietary cure, health foods, and dietary supplements. The dosage form and shape of the composition are also not particularly limited, and the composition can be used in the form of a liquid preparation such as solution, suspension or emulsion; a semi-solid preparation such as paste; granule; powdered medicine; powder; tablet; capsule (including soft and hard capsules); or an internal composition for use in capsules.

For example, when sesamin is selected as a lignan compound, the composition produced by the process of the present invention can comprise the lignan compound in a total amount of 0.001 to 90 wt. %, 0.01 to 50 wt. %, or 0.1 to 10 wt. %.

Said composition can have at least one lignan compound being mixed with an acceptable carrier or excipient. Examples of the carrier include, but are not limited to, water, physiological saline, edible oil, ethanol, propylene glycol, and glycerol. Examples of the excipient include, but are not limited to, glucose, sucrose, lactose, dextrin, cyclodextrin, xanthan gum, guar gum, gum arabic, tragacanth gum, gellan gum, locust bean gum, carrageenan, pectin, and agar. Also, additional substances commonly used in pharmaceutical formulation, such as an emulsifier, a tonicity agent, a buffer, a solubilizer, an antiseptic, a stabilizer, and/or an antioxidant, can be added as appropriate. Further, the composition can, if necessary, have mixed therewith any other additives such as mineral; vitamins like vitamin E, C and A; nutritional ingredients; flavorants; and colorants, as long as such additives do not impair the effects of a lignan compound, namely, mixing a lignan compound with such additives does not give rise to an unfavorable interaction.

Hereunder, the present invention will be more specifically described by way of working examples. However, this invention is not limited to these examples.

### EXAMPLES

### Example 1

### Effect of a Lignan Compound on the Expression Rhythm of the Per 2 Gene in Cultured Cells

The cell line used was Per2::LUC knock-in mouse-derived embryonic fibroblast line. Per2::LUC knock-in mouse is a mouse in which the luciferase gene serving as a reporter is introduced downstream of the promoter region of the clock gene Per 2. Therefore, a Per 2::LUC knock-in mouse-derived embryonic fibroblast line expresses the luciferase gene along with periodic expression of the clock gene. When Per 2::LUC knock-in mouse-derived embryonic fibroblasts are cultured in a culture medium containing luciferin, a luciferase substrate, the cells produce chemiluminescence periodically; thus, by monitoring the chemiluminescence, the expression rhythm of the clock gene Per 2 can be evaluated.

At first, about 5×10⁵ cells of the Per2::LUC knock-in mouse-derived embryonic fibroblast line were seeded onto 35 mm culture dishes, and then treated with 200 nM dexamethasone for 2 hours to thereby reset (tune) the biorhythm of the cells. Thereafter, the cells were cultured by replacing the culture medium with a medium containing the luminescent substrate luciferin and by adding the test substance, a 1:1 mixture of sesamin/episesamin, at different concentrations of 1.5 to 50 μM. While the cell culture was continued, the chemiluminescence of the reporter gene was measured real-time with a luminometer (LumiCycle; produced by Actimetrics) for one minute at a frequency of every 10 minutes, and the measurement was continued for 5 days. Amplitude and period length were calculated from the obtained waveform to evaluate the effect of the test substance, a 1:1 mixture of sesamin/episesamin. The results are shown in FIG. 2.

It was demonstrated that the test substance sesamin, particularly at concentrations ranging from 3 to 50 μM, increases the amplitude of the circadian rhythm in Per 2 gene expression in a dose-dependent manner.

### Example 2

### Effect of a Lignan Compound on the Rhythm in In Vivo Expression of the Per 2 Gene

The animals used were Per 2::LUC knock-in mice as described in Example 1. In these mice, the reporter gene, luciferin, introduced at downstream of the particular clock gene is expressed periodically, and thus chemiluminescence is produced by administering the luminescent substrate luciferin exogenously. Therefore, the expression of the clock gene Per 2 can be monitored in vivo over time.

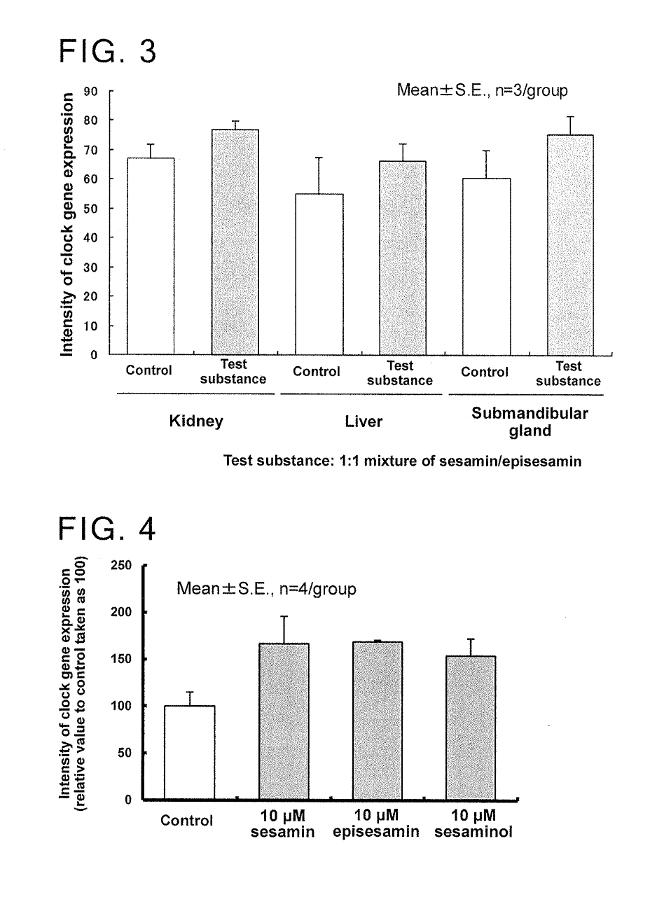

At first, the test substance, a 1:1 mixture of sesamin:episesamin, was forcibly administered orally to the Per 2::LUC knock-in mice at a dose of 250 mg/body kg per day for 3 days. Olive oil was used as a solvent control. At 3 hours after the final administration, 15 mg/kg of a luciferin solution was administered subcutaneously to mice anesthetized by isoflurane inhalation. Subsequent chemiluminescence was detected with an in vivo imaging (IVIS) kinetics system (produced by Caliper Life Sciences). Chemiluminescence detection was performed on three organs—kidney, liver and submandibular gland. Also, the chemiluminescence detection was performed a total of 6 times at a frequency of every 4 hours over a whole day after administration of the test substance, a 1:1 mixture of sesamin:episesamin, including performing the aforementioned procedure. The results are shown in FIG. 3.

It was observed that oral administration of the test substance sesamin to mice amplifies the amplitude of expression of the clock gene Per 2 in kidney, liver and submandibular gland of the mice.

### Example 3

### Effect of a Lignan Compound on the Rhythm in Expression of the Per 2 Gene in Cultured Cells—Part 2

This study was conducted by the same procedure as described above in Example 1. As test substances, sesamin, episesamin, and sesaminol were each added at a concentration of 10 μM and evaluated for the effect thereof. The results are shown in FIG. 4. The results demonstrated that sesamin, episesamin and sesaminol are all capable of increasing the amplitude of Per 2 gene expression.

1. A circadian rhythm improving composition comprising at least one lignan compound.

2. The circadian rhythm improving composition according to claim 1, wherein the lignan compound comprises sesamin, episesamin, sesamolin, sesamol, or sesaminol, or a glycoside or metabolite thereof, or a mixture of two or more thereof.

3. The circadian rhythm improving composition according to claim 1, wherein the circadian rhythm improving composition is capable of amplifying the amplitude of the expression rhythm of at least one clock gene.

4. The circadian rhythm improving composition according to claim 3, wherein the clock gene comprises Per 2 or Cry 1 gene.

5. The circadian rhythm improving composition according to claim 1, for use in the prevention, alleviation or treatment of a circadian rhythm disorder associated with aging.

6. The circadian rhythm improving composition according to claim 5, wherein the circadian rhythm disorder associated with aging is selected from the group consisting of sleep disorders, day-night reversal of behaviors, and flattening of body temperature rhythm amplitude, which are associated with aging.

7. The circadian rhythm improving composition according to claim 1, for use in the amelioration of difficulty falling asleep, decreased depth of sleep, and difficulty awakening.

8. The circadian rhythm improving composition according to claim 1, wherein the circadian rhythm improving composition is provided as an agent.

9. A process for producing a composition having circadian rhythm improving activity, the process comprising the step of formulating at least one lignan compound.

10. The process according to claim 9, wherein the lignan compound comprises sesamin, episesamin, sesamolin, sesamol, or sesaminol, or a glycoside or metabolite thereof, or a mixture of two or more thereof.

11. Use of at least one lignan compound in the production of a composition having circadian rhythm improving activity.

12. The use according to claim 11, wherein the lignan compound comprises sesamin, episesamin, sesamolin, sesamol, or sesaminol, or a glycoside or metabolite thereof, or a mixture of two or more thereof.
